(19) United States
(12) Patent Application Publication
(10) Pub. No.: US 2023/0397978 A1
(43) Pub. Date: Dec. 14, 2023

(54) IMPLANTABLE DEVICES WITH ANTIBACTERIAL COATING

(71) Applicant: University of Pittsburgh - Of the Commonwealth System of Higher Education, Pittsburgh, PA (US)

(72) Inventors: Rui Liang, Canonsburgh, PA (US); Mostafa Bedewy, McDonald, PA (US); Eric Rafeek Shaker, Marlboro, NJ (US)

(21) Appl. No.: 18/031,721

(22) PCT Filed: Oct. 13, 2021
(86) PCT No.: PCT/US2021/054724

§ 371 Date: Apr. 13, 2023

Related U.S. Application Data
(60) Provisional application No. 63/090,891, filed on Oct. 13, 2020.

(51) Int. Cl. A61F 2/00; A61L 17/14; A61L 17/00; A61L 31/08; A61L 31/04

(52) U.S. Cl. CPC A61F 2/0063; A61L 17/145; A61L 17/005; A61L 31/084; A61L 31/048; A61L 2300/404; A61L 2400/12

(57) ABSTRACT

An implantable mesh device includes a surface layer on at least a portion thereof. The surface layer includes a plurality of extending members that mechanically interact with microbiota to disable the microbiota.

### CROSS-REFERENCE TO RELATED APPLICATIONS

This application claims benefit of U.S. Provisional Patent Application Ser. No. 63/090,891, filed Oct. 13, 2020, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Implantation of indwelling medical devices has become an integral part of healthcare to save and improve quality of life thanks to the rapid advancement of technology in material science, electronics, etc. However, the implantation itself involves breaching of tissue continuity (creating wound) and introducing a foreign material into the body, both of which increase the chance of microbial infections. It is estimated that implant-associated infections account for 50-70% of the nearly 2 million nosocomial infections every year in the United States. These infections often lead to device removal and long-term aggressive antibiotic treatment, which not only eliminates any benefit arising from an implant but also poses an enormous burden on the healthcare system. This issue is exacerbated in patients whose immune defense is impaired, for example patients with diabetes. Higher infection rates and biofilm formation have been found in such patients who received implants. Diagnosis and treatment of implant-associated infections remain major obstacles. Thus, preventive interventions become particularly important to reduce the risk of infection.

Synthetic meshes, typically manufactured from polymer such as polypropylenes, are often used to augment surgical repairs using native tissue. Surgical meshes are woven or knitted sheets which are used as either a permanent or temporary support for organs and other tissues during surgery. Surgical mesh may be formed from both inorganic and biological materials and is used in a variety of surgeries. Though hernia repair surgery is the most common application, it can also be used for reconstructive purposes, such as in pelvic organ prolapse. Examples include hernia meshes to augment abdominal wall defects and urogynecologic meshes (mid-urethral slings and prolapse meshes) to augment soft tissue support in the treatment of stress urinary incontinence and pelvic organ prolapse. In the Medical Device Reports and “Safety communication” released by the FDA in 2011, infection was listed as one of the most common mesh-associated complications. Overt infection with clear local infectious symptoms and/or systemic inflammatory response syndrome occurs in approximately 10% for hernia meshes, and approximately 8% for urogynecologic meshes.

However, the rates of infection can be well underestimated due to its insidious process and a lack of clear diagnostic definition. For example, pain symptoms, mesh exposure through vaginal epithelium, and/or mesh erosion into adjacent organ cavities, which occurs at much higher frequency and are not thought to be infection, can all be secondarily related to microbial contamination. Indeed, bacterial biofilm formation is a major cause of implant infection and failure. The biofilm-based infections are subtle and always chronic in nature. The duration from microbial inoculation and surface attachment to recognizable device infection can last for weeks even years.

The typical intervention is to reduce the bacterial inoculate load when a sterile anatomic compartment is breached. Perioperative antibiotics, sterile surgical techniques, disinfection dressings, etc. have been used. While reduced infection rates have been demonstrated, implant associated infection still occurs at high frequency since the procedures cannot eradicate all the bacteria colonized at skin and mucosa. For example, even after repeated pre-surgical preparation, the vagina is still colonized with significant number of bacterial colonies. During the transvaginal procedures of mesh implantation, colonization of mesh implants has been found to occur frequently despite low bacterial densities, constituting a risk for subsequent infections.

In an attempt to reduce the risk of infection, various coatings such as metal coatings, which have potent bactericidal properties, have been applied to an implantable device surface. However, metal coatings often decay or degrade and release toxic and/or carcinogenic metal ions within the body. Recently, carbonaceous coatings have been developed for a number of surfaces which form unique topographies that can physically interact with bacterial cells to limit their attachment and growth.

Existing technologies in surface coating design/materials for medical implants attempt to balance a number of factors including effectiveness against bacteria, biodegradability, susceptibility to decay, and malleability. Materials at the forefront of implant coating technology are magnesium, gold, silver and copper coatings which are similarly effective against bacterial infection. Unfortunately, there are a number of problematic issues with all existing coating materials and/or techniques when applied to indwelling implants. Further, no suitable coatings are yet available for hernia meshes, urogynecological meshes and similar implantable devices.

### SUMMARY

In one aspect, an implantable mesh device includes a surface layer on at least a portion thereof. The surface layer includes a plurality of extending members that mechanically interact with microbiota to disable the microbiota. The surface layer may, for example, include, consist of, or be formed of graphene or a graphitic material other than graphene. In a number of embodiments, the surface layer includes, consists of, or is formed of graphene.

The plurality of extending members may, for example, include edges which are configured to interact with (for example, pierce, puncture, penetrate, cut) a membrane of a cell of the microbiota. In a number of embodiments, the edges have a width of 100 nm or less. In a number of embodiments, the edges have a width of 10 to 100 nm.

The surface layer may, for example, include interactive chemical groups to interact with therapeutic molecules or with therapeutic nanoparticles.

The implantable mesh device may, for example, include a polymeric material on a surface thereof. the polymeric material may, for example, include polypropylene. The surface layer may, for example, be formed via direct or indirect carbonization to form the surface layer on at least a portion of the polymeric material, wherein the surface layer includes or is formed of graphene. In a number of embodiments, the surface layer is formed via direct or indirect laser carbonization.

In a number of embodiment of the implantable mesh device, the mesh is a hernia mesh or a urogynecologic mesh.

In a number of embodiments, the surface layer includes interactive groups such as oxygen-containing chemical group or nitrogen-containing chemical groups. As descried above, such groups may interact with therapeutic molecules or with therapeutic nanoparticles. The therapeutic molecules or therapeutic nanoparticles may be associated with a plurality of the oxygen-containing chemical groups or a plurality of the nitrogen-containing chemical groups. In a number of embodiments, the therapeutic molecules or therapeutic nanoparticles are antimicrobial molecules.

In a number of embodiments, the surface layer includes, consists of, or is formed of graphene and the surface layer is formed via Plasma Enhanced Chemical Vapor Deposition.

In another aspect, a method of fabricating an implantable mesh device includes forming a surface layer on at least a portion of a surface of a component of the implantable mesh device. The surface layer includes a plurality of extending members that mechanically interact with microbiota to disable the microbiota. The surface layer may, for example, include, consist of, or be formed of graphene or a graphitic material other than graphene. In a number of embodiments, the surface layer includes, consists of, or is formed of graphene.

As described above, the plurality of extending members may, for example, include edges which are configured to interact with (for example, pierce, puncture, penetrate, cut) a membrane of a cell of the microbiota. In a number of embodiments, the edges have a width of 100 nm or less. In a number of embodiments, the edges have a width of 10 to 100 nm.

The surface layer may, for example, include interactive chemical groups to interact with therapeutic molecules or with therapeutic nanoparticles.

The component of the implantable mesh device may, for example, include a polymeric material on a surface thereof. The polymeric material may, for example, include polypropylene. The surface layer may, for example, be formed via direct or indirect carbonization to form the surface layer on at least a portion of the polymeric material, wherein the surface layer includes or is formed of graphene. In a number of embodiments, the surface layer is formed via direct or indirect laser carbonization.

In a number of embodiment of the implantable mesh device, the mesh is a hernia mesh or a urogynecologic mesh.

In a number of embodiments, the surface layer includes interactive groups such as oxygen-containing chemical group or nitrogen-containing chemical groups. As descried above, such groups may interact with therapeutic molecules or with therapeutic nanoparticles. The method may further include associating therapeutic molecules or therapeutic nanoparticles with a plurality of the oxygen-containing chemical groups or a plurality of the nitrogen-containing chemical groups. In a number of embodiments, the therapeutic molecules or therapeutic nanoparticles are antimicrobial molecules.

In a further aspect, an implantable device includes a polymeric material (for example on a surface a component thereof) and a surface layer on at least a portion of the polymeric material. The surface layer include a plurality of extending members that mechanically interact with microbiota to disable the microbiota. The surface layer may, for example, include, consist of, or be formed of graphene or a graphitic material other than graphene. In a number of embodiments, the surface layer includes, consists of, or is formed of graphene.

As described above, the plurality of extending members may, for example, include edges which are configured to interact with (for example, pierce, puncture, penetrate, cut) a membrane of a cell of the microbiota. In a number of embodiments, the edges have a width of 100 nm or less. In a number of embodiments, the edges have a width of 10 to 100 nm.

The surface layer may, for example, include interactive chemical groups to interact with therapeutic molecules or with therapeutic nanoparticles.

The surface layer may, for example, be formed via direct or indirect carbonization to form the surface layer on at least a portion of the polymeric material, wherein the surface layer includes or is formed of graphene. In a number of embodiments, the surface layer is formed via direct or indirect laser carbonization.

In a number of embodiments, the polymeric material includes polypropylene. In a number of embodiments, the implantable device is a mesh, a suture, a fiber, or a film.

The surface layer may, for example, include oxygen-containing chemical group, nitrogen-containing chemical groups, or other groups which interact with therapeutic molecules or therapeutic nanoparticles. Therapeutic molecules or therapeutic nanoparticles may be associated with a plurality of the oxygen-containing chemical groups, a plurality of the nitrogen-containing chemical groups, or with a plurality of other interactive chemical groups. In a number of embodiments, the therapeutic molecules or therapeutic nanoparticles are antimicrobial molecules.

In still a further aspect, a method of fabricating an implantable device, which includes a polymeric material, includes forming the implantable device and forming a surface layer on at least a portion of the polymeric material thereof. The surface layer includes a plurality of extending members that mechanically interact with microbiota to kill the microbiota. Once again, the surface layer may, for example, include, consist of, or be formed of graphene or a graphitic material other than graphene. In a number of embodiments, the surface layer includes, consists of, or is formed of graphene.

As further described above, the plurality of extending members may, for example, include edges which are configured to interact with (for example, pierce, puncture, penetrate, cut) a membrane of a cell of the microbiota. In a number of embodiments, the edges have a width of 100 nm or less. In a number of embodiments, the edges have a width of 10 to 100 nm.

The surface layer may, for example, include interactive chemical groups to interact with therapeutic molecules or with therapeutic nanoparticles.

The surface layer may, for example, be formed via direct or indirect carbonization to form the surface layer on at least a portion of the polymeric material, wherein the surface layer comprising graphene. The surface layer may, for example, be formed via direct or indirect laser carbonization. In a number of embodiments, the polymeric material includes polypropylene. In a number of embodiments, the implantable device is a mesh, a suture, a fiber, or a film.

As described above, the surface layer may, for example, include oxygen-containing chemical group, nitrogen-containing chemical groups, or other groups which interact with therapeutic molecules or therapeutic nanoparticles. Therapeutic molecules or therapeutic nanoparticles may be associated with a plurality of the oxygen-containing chemical groups, a plurality of the nitrogen-containing chemical groups, or with a plurality of other interactive chemical groups. In a number of embodiments, the therapeutic molecules or therapeutic nanoparticles are antimicrobial molecules.

The present devices, systems, methods, and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1 (in panels a-c) illustrates results from fluorescent in-situ hybridization (FISH) and confocal laser scanning microscopy (CLSM) using pan-bacteria probe (EUB338) showing multiple bacterial biofilms (indicated by arrows) on the surface of mesh fibers in polypropylene meshes with different textile properties.

FIG. 2 illustrates schematically biofilm life cycle on an implantable device.

FIG. 3 is an idealized, schematic view of an implanted device surface including an anti-biofilm layer hereof and the interaction of that layer with a bacterial cell and a human cell.

FIG. 4A illustrates schematically a cutaway view of a system for achieving plasma enhanced chemical vapor deposition or PECVD of a graphitic material on a surface of an implantable device.

FIG. 4B schematically a PECVD process for deposition of a graphitic material such as graphene on a surface of an implantable device.

FIG. 5A illustrates schematically the formation of various carbonized morphologies on a section of a polymeric surface such as a section of an implantable polymer mesh to achieve various morphologies as a function of, for example, laser fluence in laser carbonization.

FIG. 5B illustrates schematically lasing a flat polymer to produce a laser-induced nanocarbon pattern or LINC along a laser direction x, wherein z is the distance from the beam waist.

FIG. 5C illustrates, in panel a, X-ray diffraction or XRD analyses of powdered LINC formed using a power P=28 W, a speed v=500 mm/s, and z=0 mm, in panel b, experimental points representing estimated laser beam profile along the lasing direction x as well as the profile of the fitted Gaussian beam illustrating the divergence of the beam away from the beam waist, wherein the resulting beam spot in the x direction is estimated to be 2w₀ₓ=251.6 μm and, in panel c, estimated optical flux along the x direction at constant power (P=22.5 W), showing the change of beam intensity with z (that is, with changing spot size by defocusing the beam).

FIG. 5D (panels a-i) illustrates scanning electron micrograph (SEM) images of side views of representative polymer (polyimide) films lased to general laser-induced nanocarbon patterns, and panels j-n illustrates enlarged section of panels a, c, d, f, and i, respectively, panel o illustrates an enlarged section of panel n, and panel p illustrates schematically the change in morphology of the carbonized pattern as a function of laser fluence.

FIG. 6A illustrates a comparative study of the antimicrobial effect of a representative silicon dioxide surface coated with a graphene surface layer hereof (one atomic layer in thickness) and coated with a gold (Au) coating for the bacteria Staphylococcus epidermidis or S. epidermidis incubated for one hour, wherein human cells, which have 1000× to 2000× the volume of bacterial cells were unaffected.

FIG. 6B illustrates a comparative study of the antimicrobial effect of a representative silicon dioxide surface coated with a graphene surface layer hereof (one atomic layer in thickness) and coated with a gold (Au) coating for the bacteria Escherichia coli or E. coli for one hour, demonstrating up to 100% decrease in bacterial viability—p<0.001) wherein human cells, which have 1000× to 2000× the volume of bacterial cells were unaffected.

FIG. 7 illustrates Table 1 setting forth a comparison of properties of anti-biofilm surface layers hereof and metallic layers.

FIG. 8A illustrates schematically an indirect carbonization methodology to from areas of a surface layer of graphene including extending members on polymer fiber (for example, a formed of a first polymer such as polypropylene) which is placed on second polymer (for example, polyimide) film.

FIG. 8B a photograph of a substrate made of polyimide, on which a polypropylene fiber is placed, wherein lateral rastering of the laser creates the horizontal black lines so that graphene is created both on the polyimide and the fibers.

FIG. 8C illustrates a photograph of a polypropylene monofilament suture fiber with clear black deposits (within broken-line boxes) resulting from graphene bands that corresponds to the positions of the back horizontal lines in FIG. 8B.

### DETAILED DESCRIPTION

The present devices, systems, methods, and compositions, along with the attributes and attendant advantages thereof, will best be appreciated and understood in view of the following description taken in conjunction with any accompanying drawings.

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an extending member” includes a plurality of such extending members and equivalents thereof known to those skilled in the art, and so forth, and reference to “the extending member” is a reference to one or more such extending members and equivalents thereof known to those skilled in the art, and so forth. Recitation of ranges of values herein are merely intended to serve as a shorthand method of referring individually to each separate value falling within the range. Unless otherwise indicated herein, and each separate value as well as intermediate ranges are incorporated into the specification as if it were individually recited herein. All methods described herein can be performed in any suitable order unless otherwise indicated herein or otherwise clearly contraindicated by the text.

Although the coatings hereof may be used in connection with any implantable device or system, such coatings are particularly advantageous when use in connection with implantable devices or systems including meshes as well as implantable devices include one or more components having a surface comprising a polymeric material. The component(s) may, for example, be formed completely from a polymeric material or the polymeric material may be attached to or coated upon another material/substrate (for example, a metal, a glass, or another substrate).

Bacterial growth and biofilm formation on implantable mesh surfaces, which subsequently results in infection, are significant concerns for implantable mesh applications. This issue is exacerbated in immunocompromised patients (for example, diabetic patients whose immune defenses are impaired). Higher infection rates and biofilm formation have indeed been found in diabetic patients in which mesh systems have been implanted. Bacterial cells are sufficiently small to enter and colonize within the relatively small pores or interstices of an implanted mesh. While bacteria are small enough to colonize in such pores or interstices, immune cells such as neutrophils and macrophages are often too large to access such pores and interstices.

In accordance with previous studies, studies hereof showed that polymeric (for example, polypropylene) meshes are susceptible to biofilm formation. Such biofilms were easily colonized by Escherichia coli (E. coli) and covered by biofilm patches with bacteria embedded. With fluorescence in-situ hybridization, the mesh-tissue samples excised from women with mesh complications (exposure/erosion and pain) were probed for the presence of biofilm at mesh-tissue interface. It was discovered that ˜45% of samples were positive. With a strategy using laser capture microdissection to collect tissue at mesh-tissue interface (where the biofilm forms) followed by bacterial DNA sequencing, different bacterial species were recovered, most abundantly Staphylococcus, Neisseria, and Escherichia, which are well-recognized species that can form biofilms and cause implant-associated infection. Those results support that biofilm infection is a major cause of mesh-related complications. Reduction/prevention of the formation of such biofilms will substantially decrease the rate of mesh complications and consequently healthcare cost.

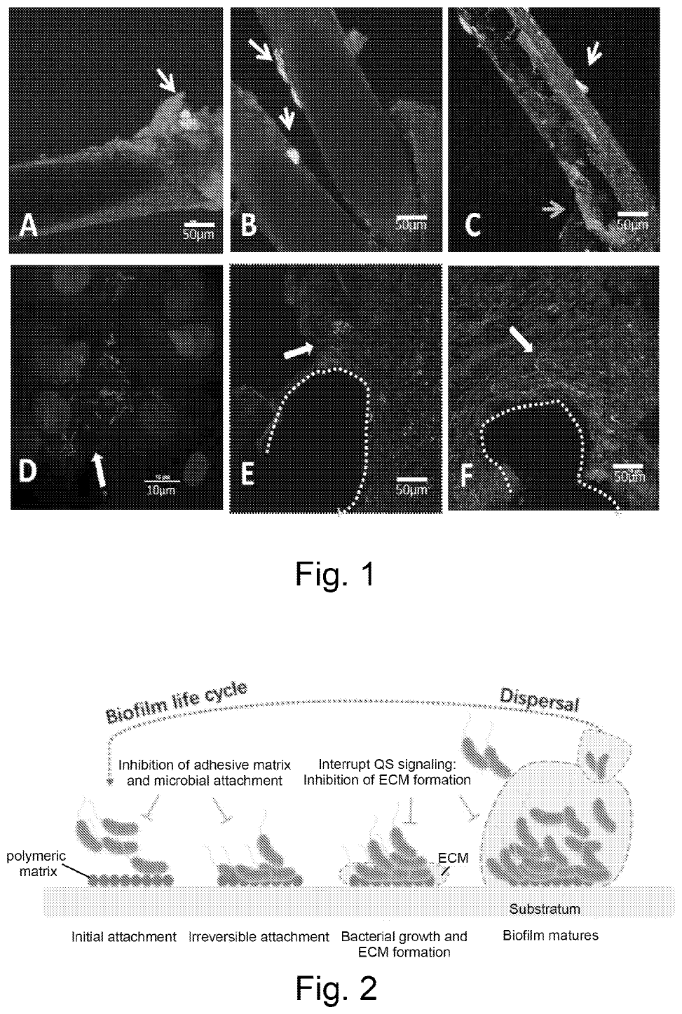

FIG. 1 (in panels A-C) illustrates results from fluorescent in-situ hybridization (FISH) and confocal laser scanning microscopy (CLSM) using pan-bacteria probe (EUB338) showing multiple bacterial biofilms (indicated by arrows) on the surface of mesh fibers in polypropylene meshes with different textile properties—Gynemesh PS (pane A), UltraPro (panel B) and Restorelle (panel C) after cultured with E. coli for 24 hours. The images are set forth at 400× magnification. Panels D-E illustrates results from FISH/CLSM showing bacterial clusters in human vaginal swab (panel D) and biofilms on the surface of mesh fibers in a mesh-tissue sample excised from a woman having mesh exposure (panel E) and pain (panel F) of a mesh fiber. A dashed or broken line marks the contours of mesh fibers with arrows pointing at bacterial clusters embedded in matrix. The images of panels D-F are set forth at 600× magnification.

FIG. 2 illustrates the biofilm life cycle on a polymeric surface or matrix of an implantable device such as a mesh. In a mature biofilm, bacterial cells crowd together and are surround by an extracellular matrix or ECM. Quorom sensing or signaling (QS) may, for example, play important roles in biofilm development. Pathogenic bacteria in biofilms may, for example, use QS mechanisms to develop antibiotic resistance and to activate virulence. In general, biofilm formation begins with a reversible attachment of bacterial cells to a surface. The bacteria subsequently form a monolayer and irreversibly attach to the surface via the formation of an ECM. With subsequent bacterial growth, a monocolony is formed including multiple layers of bacterial cells. In later stages of a mature biofilm, some bacterial cells will detach and the biofilm will disperse. Strategies to prevent the formation of biofilms and associated diseases including anti-biofilm substances, quorum quenching molecules, bio-surfactants, and competitive inhibitors.

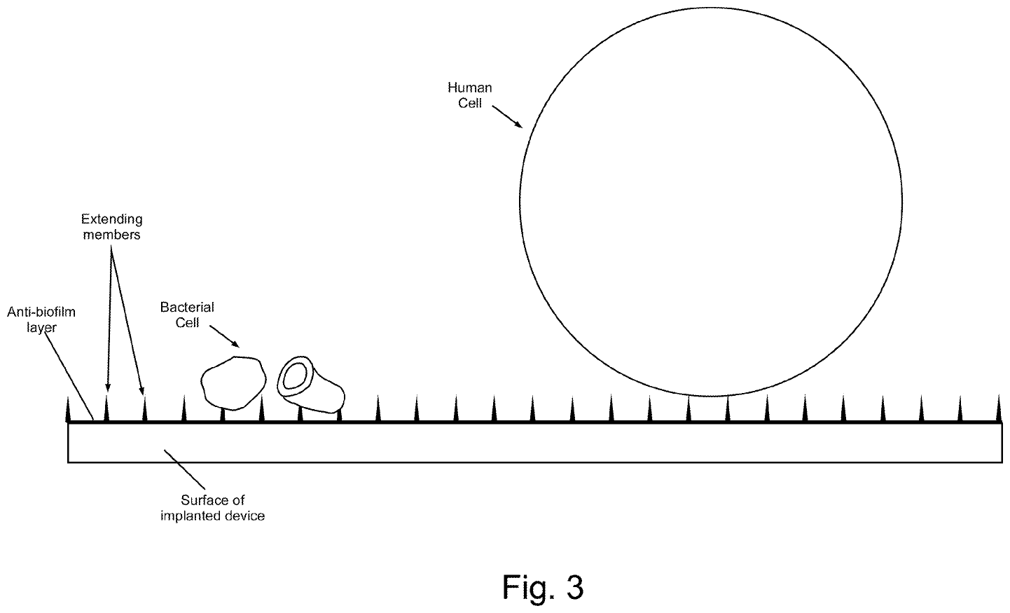

In a number of embodiments hereof, an implantable device includes a surface layer or coating on at least a portion thereof. The layer includes a plurality of extending members which mechanically interact with microbiota (for example, bacteria) to disable the microbiota. As used herein, the term “disable” when used in connection with microbiota such as bacterial refers to killing or reducing/eliminating the functionality of the microbiota (that is, reducing or eliminating the ability of the microbiota to attach to a surface and/or to proliferate). Anti-microbiota or anti-biofilm surface layers hereof may, for example, include a plurality of relatively pointed or sharp extending members that mechanically interact with microbiota to disable the microbiota. A sliced/mechanically damaged bacterial cell and an unaffected human cell are illustrated schematically in FIG. 3 in the vicinity of a surface layer hereof which includes pointed or sharp extending members. The components illustrated in FIG. 3 are not drawn to scale.

In a number of embodiments hereof, the bactericidal and/or anti-biofilm surface layers or coatings hereof are formed upon or applied to polymeric surfaces of one or more components of implants. In a number of representative embodiments, such surface layers or coatings are formed upon or applied to mesh implants which are typically fabricated from a polymeric material such as polypropylene. As described above, the anti-biofilm layers hereof include extending members that extend outward (that is, in a direction generally normal to the surface) from the surface of the implantable device. Such extending member may, for example, be oriented generally normal to the surface (that is, within 20% or within 10% of normal to the surface at a particular area thereof from which the extending member extends). The average length, height, and width (that is, the conformation/morphology) and the average number per unit surface of the extending members may be controlled to selectively interact mechanically with the membranes of microbiota/bacterial cells to disrupt, penetrate, puncture, or slice open such cells.

In a number of embodiments, the layers hereof are formed from or include a graphitic material such as graphene. Graphitic materials are carbonaceous materials including sp² hybridized carbon bonds. Graphitic materials include, for example, graphite, graphene, graphene oxide, graphite oxide, reduced graphene oxide, and reduced graphite oxide. The stability of generally normally oriented graphitic materials such as graphene makes such materials essentially impervious to decay or degradation, setting them apart from materials currently used in surface layer or coatings for implantable devices. The anti-biofilm layers hereof have the potential to save the healthcare system billions of dollars while providing patients with peace of mind regarding the reliability and safety of implants.

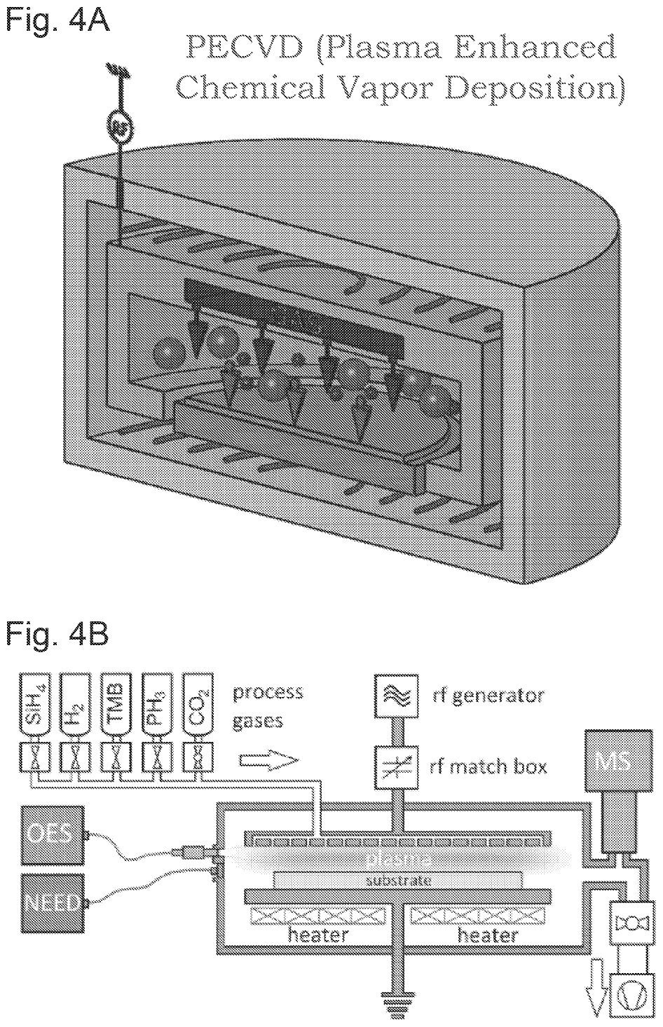

In a number of embodiments of the formation of the anti-biofilm layers hereof, the layer material (for example, a carbonaceous or graphitic material such as graphene) is applied to or formed upon a surface of a component of the implantable device surface (either after or during fabrication of the device). In general, different substrates may require different techniques. In a number of representative embodiments hereof, a graphitic layer is applied or formed using the technique of Plasma Enhanced Chemical Vapor Deposition or PECVD as illustrated in FIGS. 4A and 4B. Because of the relatively low melting temperature of certain polymers, however, certain techniques such as PECVD for surface layer/coating formation are not applicable to such polymers. For example, polypropylene which is typically used in forming medical meshes, is not directly suitable for use with PECVD. Higher-melting-temperature polymers or polymer composites may, for example, be used in connection with PECVD and other high-temperature deposition techniques.

A number of other techniques may be used in forming the surface layers hereof on polymeric surfaces such as the surfaces of polymeric medical meshes. For example, in the case of graphite nanoplatelets, spray coating or extrusion-etching of graphite composites of graphite nanoplatelets (GNP) and a polymer such as low-density polyethylene (LDPE) may be used. For the latter, GNP is first coated upon LDPE particles and the composites are extruded in controlled conditions (that is, controlled temperature, density, speed, etc.) through an extruder to obtain controlled orientation of GNP flakes within the polymer matrix. The GNP flakes may then be exposed by etching the surface of the GNP-LDPE nanocomposites.

Graphitic and/or other carbonaceous layers may also be formed on polymers (including polymer fibers of sutures or of meshes) via carbonization of the polymer as further discussed below. Representative embodiments hereof may be fabricated from a synthetic mesh product based on carbonization of polymer fibers with graphene-based morphologies. Such carbonization may, for example, occur be performed after formation/fabrication of the implant or at an intermediate stage in such formation/fabrication. Through controlling the morphology (flakes, spikes, needles and their sizes) of graphene surface layer, the implant surface will block bacterial attachment to mesh fibers, which will significantly reduce or eliminate biofilm formation by bacteria surviving peri-surgical preparation.

The term “polymer” or the prefix “poly” (when referring to a particular type of polymer) refers generally to a molecule, the structure of which includes repeat units derived, actually or conceptually, from molecules of low relative molecular mass (monomers). The term polymer includes both naturally occurring polymer such as biopolymers (for example, silk or other natural fibers) and synthetic polymers. Likewise, the term polymer includes copolymers, which include two or more different repeat units, linear polymers, branched polymers, cyclic polymers etc. In general, any polymer which is relatively rich in carbon may provide a substrate for carbonization as described herein.

The fabrication of three-dimensional graphene-based nanocarbons on polymer surfaces of implants such as individual polymer fibers making up, for example, suture or vaginal and other meshes may be achieved by either direct or indirect laser processing in a controlled gaseous environment. Laser carbonization may, for example, be carried out on a polymer surface of an implant or upon a polymer component (for example, a fiber) of the implant before assembly/formation of the implant. Carbonization (for example, laser carbonization) of polymers has not been previously used in forming anti-biofilm surface on polymeric implantable devices. Graphene-based structures/layers formed via laser carbonization are strongly adhered to the surface of the polymer. In the case of direct laser processing, direct carbonization of the polymer making up the implant (for example, the fibers of a mesh) is achieved upon laser irradiation in, for example, air, in vacuum, or in an inert gas atmosphere. In the case of indirect carbonization, a second polymer may be placed adjacent a substrate (for example, a surface of a first polymer of an implant) and the second polymer carbonized via laser carbonization wherein a resultant carbonized layer attaches to the surface of the substrate (for example, through interaction such as melt interaction/welding, chemical bonding, physical interaction, etc.).

Control of composition and/or pressure of a gaseous environment in laser carbonization may be used to control the direct transformation of polymer into graphene-based nanostructures with different morphologies. Laser parameters such as power, scanning speed, and degree of beam defocus enables creating a variety of different morphologies directly on the polymer substrate, wherein the fiber itself is the precursor for the growth of such laser-induced graphene nanostructures. The morphology of graphene protrusion can be tightly controlled displaying, for example, isotropic porous morphology, anisotropic cellular networks, aligned wooly nanofibers, as well as other outward pointing spikes. Feature sizes of sub-100-nm in penetrating surfaces may be created by this process including nanospikes branched structures and needles, which is an effective size range to penetrate bacterial cell walls. Smaller width/diameter spikes, needle or knife edges (˜10 nm) can also induce DNA fragmentations, triggering bacterial death/apoptosis.

Determining or mapping of laser conditions that lead to differences in physical quantities laser-induced nanocarbon or LINC morphologies grown on polymeric materials is, for example, described in U.S. Provisional Patent No. 63/140,463, filed Jan. 22, 2021 and assigned to the assignee of the present application, as well as in Abdulhafez, M. et al., Fluence-Dependent Morphological Transitions in Laser-Induced Graphene Electrodes on Polyimide Substrates for Flexible Devices, ACS Appl. Nano Materi., 4, 3, 2973-2986 (2021). Control of conditions is important in achieving controlled and repeatable surface patterns of predetermined morphological, chemical, and/or electrical properties. One may, for example, readily identify transitional levels, values or thresholds in processing parameters resulting in changes in morphological, chemical and/or other properties.

As described above, laser carbonization methodologies described herein provide an unprecedented capability to create compositions including patterns of graphene-based nanocarbon materials including nanofibers, branched networks and other nanoporous morphologies with spatial control of their morphology and properties through laser carbonization of substrate polymers. A wide variety of polymers may be used in carbonization procedures hereof. A nonlimiting description of representative polymers and a general description of laser induced carbonization are, for example, summarized in U.S. Pat. No. 10,505,193.

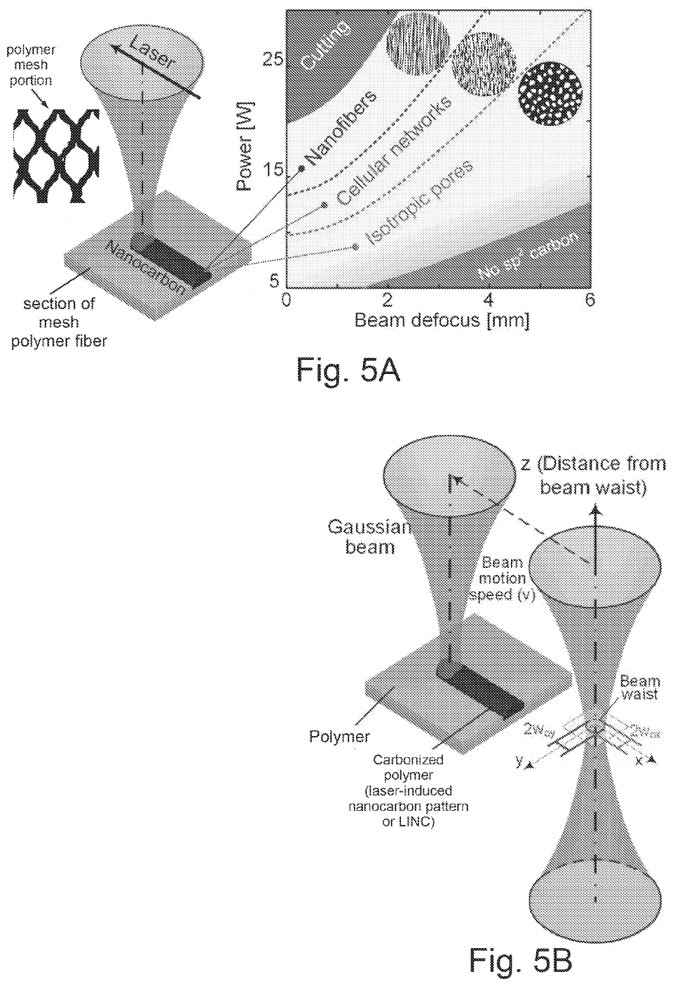

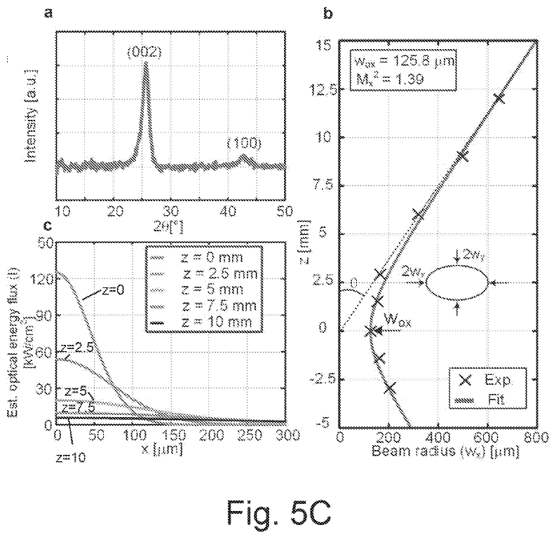

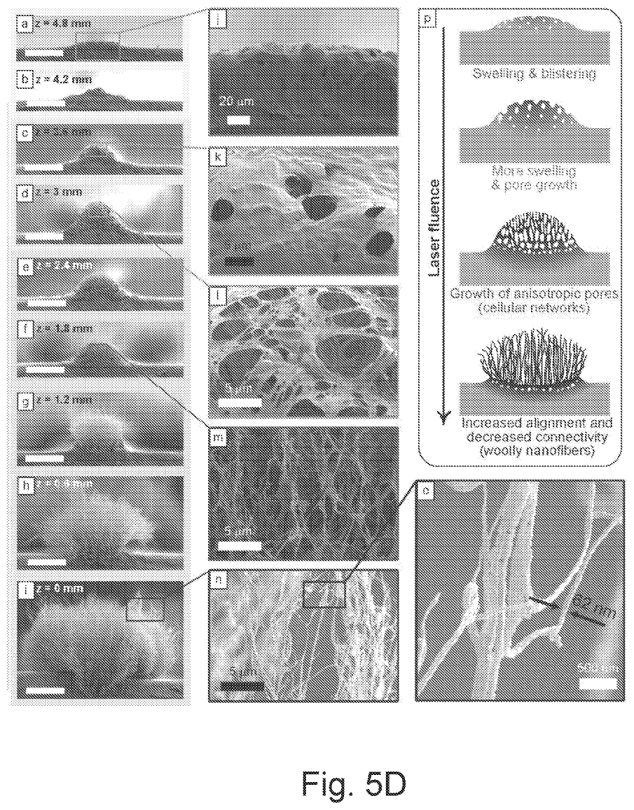

The laser induced formation of nanocarbons is a superior alternative to top-down application/printing methods that are typically employed in fabricating flexible devices with functional nanocarbon materials. A carbonized material layer may, for example, be formed on a polymeric substrate by applying a beam of electromagnetic radiation from a laser source to the polymeric substrate and controlling a fluence of the beam to or more predefined levels to control at least one property of the produced carbonized material as illustrated in FIGS. 5A through 5E. The methodology may, for example, include applying a beam of electromagnetic radiation from a laser source to a polymeric substrate, varying the position the beam over a surface of the polymeric substrate in a predetermined pattern, and controlling a fluence of the beam to one or more predefined levels to control at least one property of the produced carbonized material.

Fluence may, for example, be controlled to the one or more predefined levels by controlling a distance z between a focus or waist of the beam and a surface of the polymeric substrate, by controlling optics of the laser source or by controlling power of the laser source. In a number of embodiments, fluence is controlled to the one or more predefined levels by controlling a distance between a focus or waist of the beam and a surface of the polymeric substrate. In a number of embodiments, the one or more predefined levels are one or more threshold levels associated with a particular morphology. The one or more thresholds may, for example, include a lower threshold corresponding to an isotropic porous morphology, a higher threshold corresponding to an anisotropic cellular network and an even higher threshold corresponding to aligned nanofibers.

FIGS. 5A through 5D illustrate laser-induced carbonization processes in which control of laser fluence were achieved by controlling the vertical coordinates (Z-axis; the distance of the surface from the focus or waist of the laser beam) for points on the sample surface. A mapping of Z-values may, for example, be achieved by controlling the position of the laser source relative to the polymer surface or by controlling the optics of the laser source which focus the beam. Fluence may also be controlled by altering power such as though pulse width modulation. As described above and illustrated in FIGS. 5A and SD, a variety of nanocarbon morphologies (isotropic pores, cellular networks, and nanofibers in the illustrated embodiments) may be formed directly on, for example, the surface of a flexible polymer surface or substrate. The reason that changing Z-coordinate of the substrate surface enables creating spatial maps of laser fluence is that changing Z-values leads to controlling the degree of beam focus/defocus, which in turn controls the average fluence value on each point on the surface. Once again, control of focus/defocus may also be achieved optically. Hence, as the laser is rastered or scanned over a surface with spatially varying Z-coordinate, a spatially varying fluence map is generated, which enables creating fluence-dependent nanocarbon morphologies locally at each point of the surface. Anti-biofilm properties for a particular polymer on the basis of varied morphology and chemical composition can readily be determined via routine experimentation.

The chemical composition of the graphene-based surface layers hereof may, for example, be tuned to have a controlled chemical composition (for example, interactive chemical groups providing oxygen content, nitrogen content, etc.) so that the surface layer can more readily function as a carrier for therapeutic (for example, anti-microbial/antibacterial) chemical compounds/small molecules and/or nanoparticles, providing, for example, greater potential to perpetuate its antibacterial properties and improve host immune response to meshes. Further, functionalization of graphitic surface layers hereof by, for example, increasing the oxygen content may, for example, provide a high H₂O₂ environment adjacent to an implant surface to improve the effect of and duration of anti-bacterial properties. The chemical composition of graphene-based surface layers hereof may, for example, be tuned/controlled through control of carbonization parameters (for example, fluence, gas environment composition, pressure etc.) as well as via the selection or control of the chemical structure of the polymer being carbonized.

Once again, the carbonization process described herein is not limited to a specific type of polymer materials that can be used to, for example, fabricate meshes or other implants. Indeed, as a result of certain limitations of polypropylene meshes (including in-vivo degradation, long-lasting foreign body reaction and instability of knitted mesh configuration), new mesh materials are under development. Thus, the methodology hereof is applicable to any newly developed meshes to reduce or prevent biofilm-associated complications.

Through the procedures described above, an implantable device surface is formed with a surface layer of, for example, a graphitic/graphene material including extending members having a narrow, sharp or pointed upper edge (for example, a spike-like, needle-like, or knife-like morphology or conformation) that is functional to disrupt, penetrate, pierce or slice microbiota/bacterial cell membranes and biofilms while allowing human cells to remain intact. In general, the dimensions (particularly height) of the extending members hereof is too small to adversely affect human cell membranes. In that regard, human cells are approximately 1,000-2000 times larger in volume than single-celled bacterial organisms.

The morphology, including, for example, average length (that is, the dimension extending generally normally from a substrate surface) and surface density of the extending members can, for example, be readily controlled via control of laser carbonization as described above and/or via additional laser processing methodologies. Height may, for example, be controlled to be in the range of 40 to 5000 nm, 40 to 2000 nm, 100 to 1000 nm, or 100 to 500 nm. The averages width of the extending or piercing edge of the extending members may, for example, be no greater than 100 nm. In a number of embodiments, the average width is in the range of 10 to 100 nm. As described above, the extending member is spiked, needle-like or knife-like to achieve the best microbiota/bacteria-disabling effect. The average surface density of the extending members (that is, the average number of the extending member per unit surface area) may, for example, be readily controlled via control of laser carbonization as described above and/or via additional laser processing methodologies. Although even a single extending member will provide anti-microbiota functionality, the average surface density is desirably sufficiently high to provide sufficient contact between one or more of the extending members and microbiota cells, which are typically 1 to 2 μm in length, to significantly reduce or prevent biofilm formation. In a number of embodiments, such average surface density is at least 10 extending members per μm². One can readily optimize length, width and/or average surface density for a given application via routine experimentation.

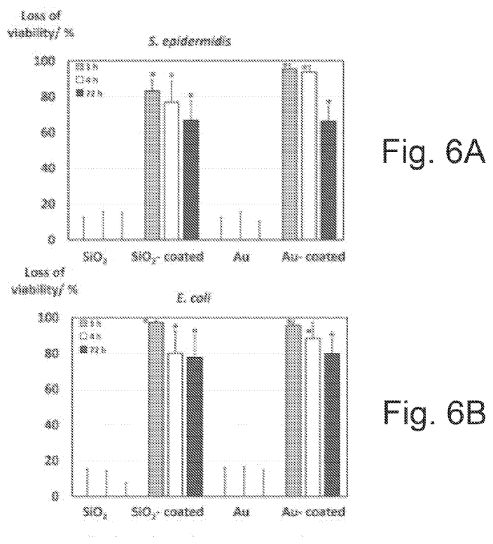

The bactericidal properties of graphene surface features or coatings including extending members have been tested and demonstrated (see, for example, FIGS. 6A and 6B). Furthermore, the bacterial cells exhibit no signs of developing resistance to the surface. Moreover, when tested on mouse fibroblasts and human neuroblastoma, no damage was done to the membranes of human cells. Unlike currently available anti-microbiota coatings, surface layers or coatings hereof (for example, formed of graphene or other graphitic material) are very stable. Unlike bactericidal metal coatings, non-metal surface layers or coatings hereof do not readily decay or degrade to release harmful ions. Advantages of surface layers or coatings hereof include, but are not limited to, bactericidal properties, mechanical disabling/destruction of bacterial cell membranes (without regard to species), prevention of biofilm formation, malleability and non-toxicity. As discussed above, although the surface layers/coatings hereof may be used in connection with many implantable devices including, for example, polymeric surfaces, such coatings/surfaces are particularly well adapted for use in connection with, for example, mesh implants (including urogynecological mesh implants and hernia mesh implants). The coatings/surfaces hereof are, for example, also well suited for use in dialysis catheters and cardiac implants (for example, left ventricular assist devices or LVADs, prosthetic heart valves, etc.).

As described above, the bactericidal effect of generally vertically extending graphitic materials/graphene is mechanical and nonspecific, meaning it will disable microbiota/bacteria regardless of their species. This feature makes the anti-biofilm surface layers hereof superior to anti-biotic or chemical coating techniques that are usually limited by their anti-bacterial spectrum, and/or chemical mechanisms regarding effectiveness, antibiotic/drug resistance and side effect, etc. Furthermore, graphene can reversibly interact with water insoluble molecules, making it a promising carrier system for sustained release of compounds/small molecule to enhance its anti-bacterial time frame and/or improve host immune reactions to the mesh implants (foreign bodies).

In a number of embodiments, an implantable device hereof includes a synthetic mesh which is coated with a graphene/graphitic surface layer/coating including a plurality of extending members which extend from the surface in a direction generally normal to the surface that interact with microbiota to prevent implant-associated infections as described above. Such meshes are typically made from a polymeric material such as polypropylene and are commonly used medical devices in patients with hernia or pelvic floor disorders such as stress urinary incontinence and pelvic organ prolapse. As described above, bacterial biofilm formation on the mesh surface is reduced/prevented by the surface layers/coatings hereof. As further described above, the unique surface layers/coatings hereof prevent/eliminate bacteria in small pores of the mesh regardless of the species of microbiota/bacteria. Pores having dimensions less than 0 μm provide beds for bacterial proliferation and persistent infection because macrophages and neutrophils are unable to enter such pores. Pores larger than 1000 μm are preferred to prevent limitation of the immune response. Of note, current products of medical meshes are knitted, which generates small and narrow spaces (˜1-4 um) at knots despite advertised large mesh pores. Once again, such narrow spaces are sufficiently large for bacteria to colonize but too small for immune cells such as neutrophils and macrophages to access. Consequently, biofilms will form without proper immune surveillance. With a surface layer of graphene including extending member having a piercing or penetrating surface having a dimension within nanometer scale, the biofilm formation in, for example, mesh knots will be efficiently prevented.

The surface layers hereof provide an anti-infection effect comparable to that of metal coatings such as magnesium and gold coatings while offering security and harmlessness, which are significant concerns when using magnesium and gold coatings. Research has shown that magnesium and gold coatings both readily corrode inside the body. The resulting toxicity of the released ions and poor wear resistance make such materials significantly less desirable for use in long-term implanted devices, such as meshes. Coating hereof in a number of embodiments include no metal.

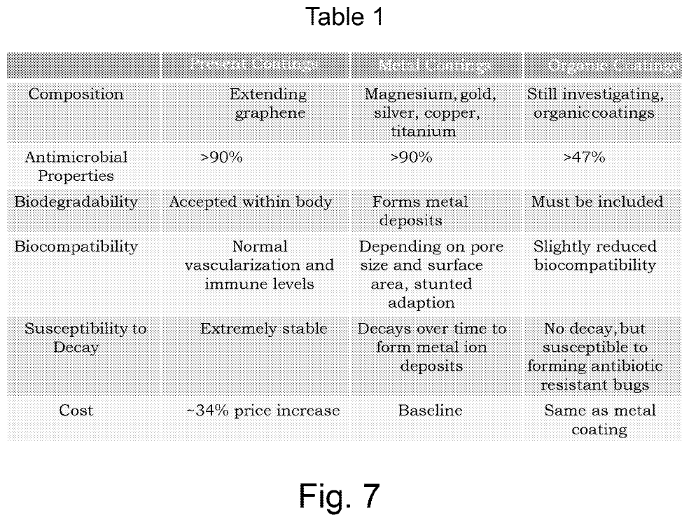

Compared to other popular alternative metal coatings such as silver and copper, which effect antimicrobial and antibacterial functions through release of silver and copper ions, respectively, the surface layers or coatings hereof are operative to disable bacteria and other microbiota by mechanical interaction. The free metal ions of silver and copper, although powerful in antimicrobial effect, also are known to cause chromosomal aberrations and mitochondrial dysfunction. A graphene or other graphitic surface layer or coating, on the other hand, does not significantly break down or degrade in-vivo and does not exhibit such side effects. Moreover, even if degradation of a graphene/graphitic coating hereof should occur (for example, enzymatically over very long periods of time), no free ions are released. A summary comparison of the surface layers/coatings hereof to metal and organic coatings currently used in connection with implantable devices is set forth in Table 1 of FIG. 7.

The market for medical implants is currently approximately 116 billion US dollars and is growing. The surface layers or coatings hereof provide a significant improvement in such medical devices.

### Experimental Examples

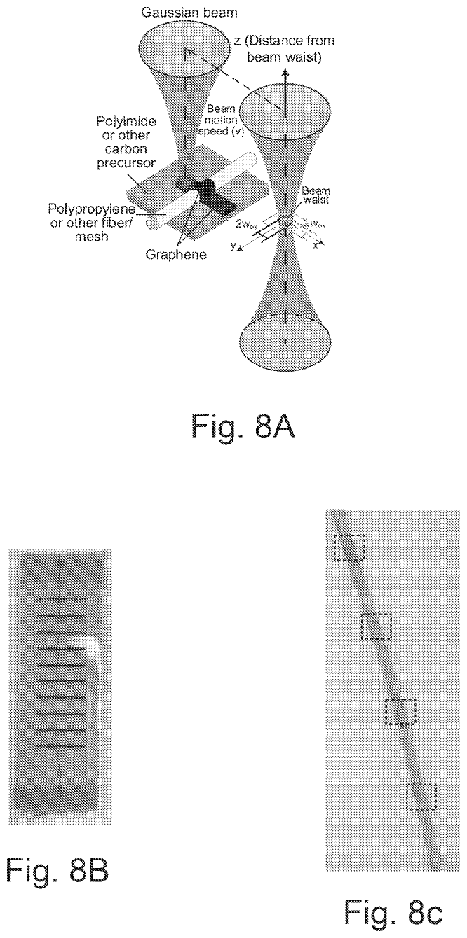

In this example, graphene nanostructures with different morphologies are fabricated on individual polymer fibers, such as polypropylene by laser irradiation, as shown schematically in FIG. 8A. In the illustrated embodiment, a substrate on which the polymer fiber, or a mesh formed of such polymer fibers, is placed can be selected to enhance the generation of graphene. For example, if the substrate underneath the polymer fiber, or mesh, is made of a polyimide or other carbon precursor material with high yield, graphene can be created on the fiber either by direct carbonization of the polymer making up the fiber or by transferring the graphene generation on the underlying substrate (for example, polyimide) to the fiber, which is heated up (or locally melted) by laser irradiation. FIG. 8B illustrates the results of this process wherein a polypropylene fiber (such as monofilament suture) is placed on polyimide for lasing across the fiber.

An important aspect of this process is that the fiber can either directly sit on the substrate with no gap between them, or the gap could be controlled to tailor the morphology of the resulting graphene. In that case, the fluence at the z-level where the fiber exists can be different from that at the z-level of the underlying substrate (that is, the degree of defocus can be different for each of the polymer fiber and the underlying substrate). Moreover, the gas environment and pressure can be chosen or controlled to further control the carbonization process and the morphology/chemical composition of the resulting graphene. FIG. 8C illustrates a photograph of a polypropylene monofilament suture fiber with clear black deposits (within broken-line boxes) resulting from graphene bands that corresponds to the positions of the back horizontal lines in FIG. 8B.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. An implantable mesh device, comprising: a surface layer on at least a portion thereof, the surface layer comprising a plurality of extending members that mechanically interact with microbiota to disable the microbiota.

2. The implantable mesh device of claim 1 wherein the surface layer comprises graphene or of a graphitic material other than graphene.

3. The implantable mesh device of claim 2 wherein the plurality of extending members comprise edges which are configured to interact with a membrane of a cell of the microbiota.

4. The implantable mesh device of claim 3 wherein the edges have a width of 100 nm or less.

5. The implantable mesh device of claim 3 wherein the edges have a width of 10 to 100 nm.

6. The implantable mesh device of claim 3 wherein the surface layer comprises interactive chemical groups to interact with therapeutic molecules or therapeutic nanoparticles.

7. The implantable mesh device of claim 1 wherein the implantable mesh device comprises a polymeric material.

8. The implantable mesh device of claim 7 wherein the surface layer is formed via direct or indirect carbonization to form the surface layer on at least a portion of the polymeric material, the surface layer comprising graphene.

9. The implantable mesh device of claim 7 wherein the surface layer is formed via direct or indirect laser carbonization.

10. The implantable mesh device of claim 8 wherein the polymeric material comprises polypropylene.

11. The implantable mesh device of claim 8 wherein the mesh is a hernia mesh or a urogynecologic mesh.

12. The implantable mesh device of claim 8 wherein the surface layer comprises oxygen-containing chemical groups or nitrogen-containing chemical groups.

13. The implantable mesh device of claim 12 wherein therapeutic molecules or therapeutic nanoparticles are associated with a plurality of the oxygen-containing chemical groups or a plurality of the nitrogen-containing chemical groups.

14. The implantable mesh device of claim 13 wherein the therapeutic molecules or therapeutic nanoparticles are antimicrobial molecules.

15. The implantable device of claim 1 wherein the surface layer comprises graphene and is formed via Plasma Enhanced Chemical Vapor Deposition.

16. A method of fabricating an implantable mesh device, comprising: forming a surface layer on at least a portion of a surface of a component of the implantable mesh device, the surface layer comprising a plurality of extending members that mechanically interact with microbiota to disable the microbiota.

17. The method of claim 16 wherein the surface layer is formed of graphene or of a graphitic material other than graphene.

18. The method of claim 17 wherein the plurality of extending members comprise edges which are configured to interact with a membrane of a cell of the microbiota.

19. The method of claim 18 wherein the edges have a width of 100 nm or less.

20. The method of claim 19 wherein the edges have a width of 10 to 100 nm.

21. The method of claim 17 wherein the surface layer comprises interactive chemical groups to interact with therapeutic molecules or therapeutic nanoparticles.

22. The method of claim 16 wherein the component of the implantable mesh device comprises a polymeric material.

23. The method of claim 22 wherein the surface layer is formed via direct or indirect carbonization to form the surface layer on at least a portion of the polymeric material, the surface layer comprising graphene.

24. The method of claim 22 wherein the surface layer is formed via direct or indirect laser carbonization of the at least a portion of the mesh.

25. The method of claim 23 wherein the polymeric material comprises polypropylene.

26. (canceled)

27. (canceled)

28. The method of claim 23 wherein the surface layer comprises oxygen-containing chemical groups or nitrogen-containing chemical groups and the method further comprising associating therapeutic molecules or therapeutic nanoparticles with a plurality of the oxygen-containing chemical groups or a plurality of the nitrogen-containing chemical groups.

29. (canceled)

30. An implantable device, comprising: a polymeric material, a surface layer on at least a portion of the polymeric material comprising a plurality of extending members that mechanically interact with microbiota to disable the microbiota.

31-55. (canceled)
